SEQUENCE LISTING (14 sequences)

SEQ ID NO: 1 - DNA, 20 bp, Bacteriophage T7
  promoter (1)..(20)
taatacgactcactataggg

SEQ ID NO: 2 - DNA, 20 bp, Bacteriophage T3
  promoter (1)..(20)
aattaaccctcactaaaggg

SEQ ID NO: 3 - DNA, 22 bp, Bacteriophage SP6
  promoter (1)..(22)
aatttaaggtgacactatagaa

SEQ ID NO: 4 - DNA, 28 bp, Artificial
  OligodT Sequence Specific RT Primer
tacaaggcaatttttttttttttttttv

SEQ ID NO: 5 - DNA, 21 bp, Artificial
  Random Sequence Specific RT Primer
  misc_feature (13)..(21): n is a, c, g, or t
tacaaggcaattnnnnnnnnn

SEQ ID NO: 6 - DNA, 30 bp, Artificial
  T7 RNA Polymerase Promoter Template
cactaatacgactcactatagggagaaatt

SEQ ID NO: 7 - DNA, 33 bp, Artificial
  RNA/DNA Composite Bridge Oligonucleotide
  misc_feature (1)..(12): RNA
  misc_feature (13)..(33): DNA
  misc_feature (33)..(33): 3′ Amino Modifier
uagggagaaauucgacacaaaaaaaaaaaaaaa

SEQ ID NO: 8 - DNA, 37 bp, Artificial
  T7/T3 RNA Polymerase Promoter Template
taatacgactcactatagggagaaattaaccctcact

SEQ ID NO: 9 - DNA, 37 bp, Artificial
  RNA/DNA Composite Bridge Oligonucleotide
  misc_feature (1)..(16): RNA
  misc_feature (17)..(37): DNA
  misc_feature (37)..(37): 3′ Amino Modifier
gaaauuaacccucacuaaagggatttttttttttttt

SEQ ID NO: 10 - DNA, 12 bp, Artificial
  Sequence Specific RT Primer
tacaaggcaatt

SEQ ID NO: 11 - DNA, 22 bp, Artificial
  T3 Promoter Oligonucleotide
gaaattaaccctcactaaaggg

SEQ ID NO: 12 - DNA, 19 bp, Artificial
  OligodT RT primer
ttttttttttttttttttv

SEQ ID NO: 13 - DNA, 9 bp, Artificial
  random RT primer
  misc_feature (1)..(9): n is a, c, g, or t
nnnnnnnnn

SEQ ID NO: 14 - DNA, 48 bp, Artificial
  3′ PolydT Test Oligonucleotide
ttctcgtgttccgtttgtactctaaggtggattttttttttttttttt

(19) United States
(12) Patent Application Publication
(10) Pub. No.: US 2007/0048741 A1
(43) Pub. Date: Mar. 1, 2007

(54) Methods and kits for sense RNA synthesis

(76) Inventors: Robert C. Getts, Collegeville, PA (US); Kelly Sensinger, Perkasie, PA (US)

Correspondence Address: Datascope Investment Corp., 14 Philips Parkway, Montvale, NJ, 07645, US

(21) Appl. No.: 11/210,602

(22) Filed: Aug. 24, 2005

(51) Int. Cl. C12Q 1/68; C12P 19/34

(52) U.S. Cl. 435/6; 435/91.2

(57) ABSTRACT

Methods and kits are provided for performing sense RNA synthesis. The sense RNA molecules can be used in various research and diagnostic applications, such as gene expression studies involving nucleic acid microarrays.

### FIELD OF THE INVENTION

The present invention relates generally to compositions and methods for synthesizing nucleic acid molecules.

### BACKGROUND OF THE INVENTION

Microarray technology has become a powerful tool for generating and analyzing gene expression profiles. Microarray expression analysis, however, generally demands large amounts of RNA that are often not available (see Wang et al., BioTechniques 34:394-400 (2003)). Several RNA amplification techniques have been developed to overcome this problem. These techniques, however, generally suffer from a phenomenon known as amplification bias (see, e.g., U.S. Pat. No. 6,582,906). In these cases, the amplified population of RNA molecules does not proportionally represent the population of RNA molecules existing in the original sample.

For example, in the method disclosed by Eberwine and colleagues (see, e.g., Van Gelder et al., Proc. Natl. Acad. Sci. USA 87:1663 (1990); U.S. Pat. Nos. 5,545,522; 5,716,785; 5,891,636; 5,958,688; and 6,291,170), a compound oligonucleotide is utilized for the amplification, wherein the compound oligonucleotide is provided with both a T7 promoter and a primer. A cDNA copy is created of an initial mRNA transcript using the compound oliognucleotide, with subsequent second strand synthesis to create a cDNA that is double stranded. RNA amplification is conducted via the promoter portion of the compound oligonucleotide, with transcription proceeding off of the cDNA's second strand. Since the second strand is used for transcription, the Eberwine method produces amplified RNA that is antisense to the initial mRNA sequence.

The Eberwine method, however, introduces a 3′ bias during each of its steps due to the incomplete processivities (i.e., the inability of an enzyme to remain attached to a nucleic acid molecule) of the enzymes utilized and the positioning of the RNA polymerase promoter (see, e.g., U.S. Pat. No. 6,582,906 and U.S. Patent Publication No. US2003/0104432). For example, the compound oligonucleotide used to produce first strand cDNA places the promoter at the 5′ end of the cDNA, which corresponds to the 3′ end of the message. This coupled with the inability of RNA polymerase to complete transcription of some templates (due perhaps to long polyA tail regions or interference from secondary and tertiary structures in the template) can result in a 3′ bias in the amplified antisense RNA population. In addition, if second strand cDNA synthesis by DNA polymerase is incomplete, these cDNAs will lack functional promoters, resulting in a reduced representation of the original RNA molecule (or possibly a complete absence) in the amplified population.

Applicants' co-pending U.S. patent application Ser. No. 10/979,052, specifically incorporated herein by reference in its entirety, discloses methods for attaching a single stranded promoter template which is not extendable with DNA polymerase comprising a RNA polymerase recognition sequence directly to the 3′ ends of first-round cDNA molecules. Following enzymatic conversion of the promoter template into a double stranded promoter with DNA polymerase, in vitro transcription is initiated by addition of RNA polymerase, resulting in the synthesis of sense RNA (sRNA) molecules having the same orientation as the original RNA molecules. Additional rounds of sRNA synthesis can be performed by reverse transcribing the sRNA molecules and re-attaching promoter templates to the second-round cDNA molecules, with subsequent enzymatic conversion into double-stranded promoters, followed by a second round of in vitro transcription with RNA polymerase.

Similarly, Applicants' co-pending U.S. patent application Ser. No. 11/150,794, specifically incorporated herein by reference in its entirety, discloses methods for attaching a single stranded promoter template which is not extendable with DNA polymerase comprising a first RNA polymerase recognition sequence and at least a second different RNA polymerase recognition sequence directly to the 3′ end of first-round cDNA molecules. Following enzymatic conversion of the promoter template into a first double stranded promoter and at least a second different double stranded promoter with DNA polymerase, in vitro transcription is initiated by addition of RNA polymerase which recognizes the first promoter, resulting in the synthesis of sRNA molecules. Additional rounds of sRNA synthesis can be performed by reverse transcribing the sRNA molecules into second-round cDNA molecules and annealing a single stranded promoter oligonucleotide complementary to the second different RNA polymerase recognition sequence to form a second double stranded promoter and initiating RNA transcription using an RNA polymerase which recognizes the second promoter. The use of a promoter template having two or more different RNA polymerase recognition sequences allows for multiple rounds of sRNA synthesis without the need for re-attachment of single stranded promoter templates and subsequent enzymatic conversion into double stranded promoters following each successive round of cDNA synthesis.

There is, however, a continuing need to provide methods for synthesizing sRNA molecules, particularly methods having a low incidence of non-specific amplification.

### SUMMARY OF THE INVENTION

Applicants have invented methods and kits for the synthesis of sRNA molecules from various nucleic acid templates, wherein a single stranded promoter template comprising at least one RNA polymerase recognition sequence is attached to the 3′ end of first-round cDNA molecules via a RNA/DNA composite bridge oligonucleotide. Applicants have discovered that the use of such a system provides robust sRNA amplification with minimal non-specific amplification.

Accordingly, one aspect of the present invention is directed to a method for synthesizing at least one sRNA molecule, comprising: providing at least one single stranded cDNA molecule having a 5′ end and a 3′ end; attaching an oligodeoxynucleotide tail onto the 3′ end of said cDNA molecule; annealing to said oligodeoxynucleotide tail a single stranded RNA/DNA composite bridge oligonucleotide comprising a 5′ RNA portion and a 3′ DNA portion, such that the RNA portion remains single stranded; extending the 3′ end of said oligodeoxynucleotide tail, such that said single stranded RNA portion becomes a double stranded RNA/DNA duplex; degrading the RNA portion of said RNA/DNA duplex, thereby exposing a 3′ single stranded DNA tail; annealing to said 3′ single stranded DNA tail a single stranded promoter template comprising at least one RNA polymerase recognition sequence; extending said 3′ single stranded DNA tail such that said at least one single stranded RNA polymerase promoter template is converted into at least one RNA polymerase promoter; and initiating RNA transcription using an RNA polymerase which recognizes said at least one RNA polymerase promoter, thereby synthesizing at least one sRNA molecule.

Another aspect of the present invention is directed to a method for synthesizing at least one sRNA molecule, comprising: providing at least one single stranded cDNA molecule having a 5′ end and a 3′ end; annealing to the 3′ end of said cDNA molecule a single stranded RNA/DNA composite bridge oligonucleotide comprising a 5′ RNA portion and a 3′ DNA sequence portion, such that the RNA portion remains single stranded; extending the 3′ end of said cDNA molecule, such that said single stranded RNA portion becomes a double stranded RNA/DNA duplex; degrading the RNA portion of said RNA/DNA duplex, thereby exposing a 3′ single stranded DNA tail; annealing to said 3′ single stranded DNA tail a single stranded promoter template comprising at least one RNA polymerase recognition sequence; extending said 3′ single stranded DNA tail such that said at least one single stranded RNA polymerase promoter template is converted into at least one RNA polymerase promoter; and initiating RNA transcription using an RNA polymerase which recognizes said at least one RNA polymerase promoter, thereby synthesizing at least one sRNA molecule.

Another aspect of the present invention is directed to a method for performing multiple rounds of synthesis of at least one sRNA molecule, comprising: providing at least one first round single stranded cDNA molecule having a 5′ end and a 3′ end; attaching an oligodeoxynucleotide tail onto the 3′ end of said first round cDNA molecule; annealing to said oligodeoxynucleotide tail a single stranded RNA/DNA composite bridge oligonucleotide comprising a 5′ RNA portion and a 3′ DNA portion, such that the RNA portion remains single stranded; extending the 3′ end of said oligodeoxynucleotide tail, such that said single stranded RNA portion becomes a double stranded RNA/DNA duplex; degrading the RNA portion of said RNA/DNA duplex, thereby exposing a 3′ single stranded DNA tail; annealing to said 3′ single stranded DNA tail a single stranded promoter template comprising a first RNA polymerase recognition sequence and at least a second different RNA polymerase recognition sequence 3′ to said first recognition sequence; extending said 3′ single stranded DNA tail such that said single stranded promoter template is converted into a first RNA polymerase promoter and at least a second RNA polymerase promoter 3′ to said first promoter; initiating a first round of RNA transcription using an RNA polymerase which recognizes said first RNA polymerase promoter to produce at least one first round sRNA molecule; synthesizing at least one second round single stranded cDNA molecule having 5′ and 3′ ends from said first round sRNA molecule, thereby forming a double stranded sRNA/cDNA duplex; degrading the sRNA portion of said sRNA/cDNA duplex leaving said second round single stranded cDNA molecule; annealing a single stranded promoter oligonucleotide complementary to said second different RNA polymerase recognition sequence of said second round single stranded cDNA molecule such that a second RNA polymerase promoter is formed; and initiating a second round of RNA transcription using an RNA polymerase which recognizes said second RNA polymerase promoter to produce at least one second round sRNA molecule, thereby performing multiple rounds of synthesis of at least one sRNA molecule.

Another aspect of the present invention is directed to a method for performing multiple rounds of synthesis of at least one sRNA molecule, comprising: providing at least one first round single stranded cDNA molecule having a 5′ end and a 3′ end; attaching an oligodeoxynucleotide tail onto the 3′ end of said first round cDNA molecule; annealing to said oligodeoxynucleotide tail a single stranded RNA/DNA composite bridge oligonucleotide comprising a 5′ RNA portion and a 3′ DNA portion, such that the RNA portion remain single stranded; extending the 3′ end of said oligodeoxynucleotide tail, such that said single stranded RNA portion becomes a double stranded RNA/DNA duplex; degrading the RNA portion of said RNA/DNA duplex, thereby exposing a 3′ single stranded DNA tail; annealing to said 3′ single stranded DNA tail an excess of a single stranded promoter template comprising a first RNA polymerase recognition sequence and at least a second different RNA polymerase recognition sequence 3′ to said first recognition sequence; extending said 3′ single stranded DNA tail such that said single stranded promoter template is converted into a first RNA polymerase promoter and at least a second RNA polymerase promoter 3′ to said first promoter; initiating a first round of RNA transcription using an RNA polymerase which recognizes said first RNA polymerase promoter to produce at least one first round sRNA molecule; synthesizing at least one second round single stranded cDNA molecule having 5′ and 3′ ends from said first round sRNA molecule, thereby forming a double stranded sRNA/cDNA duplex; degrading the sRNA portion of said sRNA/cDNA duplex leaving said second round single stranded cDNA molecule; annealing said excess single stranded promoter template to the 3′ end of said second round single stranded cDNA molecule; extending the 3′ end of said second round cDNA such that said excess promoter template is converted into a first RNA polymerase promoter and at least a second RNA polymerase promoter 3′ to said first promoter; and initiating a second round of RNA transcription using an RNA polymerase which recognizes said first or second RNA polymerase promoter to produce at least one second round sRNA molecule, thereby performing multiple rounds of synthesis of at least one sRNA molecule and producing multiple sRNA copies.

In some embodiments, polyA tails are added to the resulting sRNA molecules to increase the number and type of downstream assays in which the sRNA molecules can be used. Preferably, the sRNA molecules are reverse transcribed into cDNA molecules for use in downstream assays.

The initial single stranded cDNA molecules can be provided by contacting a RNA molecule with a primer in the presence of a reverse transcriptase. Such reverse transcription primers include oligodT primers, random primers, or combinations thereof. In some embodiments, the reverse transcription primer comprises a 5′ extension containing a specific nucleotide sequence. In other embodiments, the 3′ terminal nucleotide of the reverse transcription primer is a nucleotide or nucleotide analog that is not a substrate for terminal deoxynucleotide transferase but can be extended by reverse transcriptase. In preferred embodiments, the reverse transcription primer comprises a 5′ extension containing a specific nucleotide sequence, wherein the 3′ terminal nucleotide of the reverse transcription primer is a ribonucleotide.

Another aspect of the present invention is directed to a kit for synthesizing at least one sRNA molecule, comprising: a single stranded promoter template comprising at least one RNA polymerase recognition sequence; a single stranded RNA/DNA composite bridge oligonucleotide comprising a 5′ RNA portion and a 3′ DNA portion; and instructional materials for synthesizing sRNA molecules using said promoter template and said RNA/DNA composite bridge oligonucleotide.

Another aspect of the present invention is directed to a kit for performing multiple rounds of synthesis of at least one sRNA molecule, comprising: a single stranded promoter template comprising a first RNA polymerase recognition sequence and at least a second different RNA polymerase recognition sequence 3′ to said first recognition sequence; a single stranded RNA/DNA composite bridge oligonucleotide comprising a 5′ RNA portion and a 3′ DNA portion; a single stranded promoter oligonucleotide complementary to said second different RNA polymerase recognition sequence; and instructional materials for performing multiple rounds of synthesis of at least one sRNA molecule using said promoter template, said RNA/DNA composite bridge oligonucleotide and promoter oligonucleotide.

In some embodiments, the kits further comprise: a reverse transcriptase; an enzyme for attaching a 3′ oligodeoxynucleotide tail onto DNA molecules; an enzyme for degrading RNA in RNA/DNA duplexes; and one or more RNA polymerases.

### BRIEF DESCRIPTION OF THE DRAWINGS

FIGS. 1a-1i is a schematic representation that depicts an embodiment according to the methods of the present invention.

FIG. 2 is a photograph of a gel demonstrating RNA polymerase promoter synthesis by the methods of the present invention.

### DETAILED DESCRIPTION OF THE INVENTION

The present invention relates to methods and kits for the synthesis of sRNA molecules. The terms “sRNA molecule,” “RNA molecule,” “DNA molecule,” “cDNA molecule” and “nucleic acid molecule” are each intended to cover a single molecule, a plurality of molecules of a single species, and a plurality of molecules of different species. The methods generally comprise attaching an oligodeoxynucleotide tail onto the 3′ end of at least one cDNA molecule; annealing to the oligodeoxynucleotide tail a single stranded RNA/DNA composite bridge oligonucleotide comprising a RNA sequence 5′ of a DNA sequence, such that all of the RNA sequence and at least a portion of the DNA sequence remain single stranded; extending the 3′ end of the oligodeoxynucleotide tail, such that the portion of single stranded DNA becomes double stranded DNA and the single stranded RNA becomes a double stranded RNA/DNA duplex; degrading the RNA portion of the RNA/DNA duplex to expose a 3′ single stranded DNA tail; annealing to the 3′ single stranded tail a single stranded promoter template comprising at least one RNA polymerase recognition sequence; extending the 3′ single stranded tail such that the single stranded RNA polymerase promoter template is converted into at least one RNA polymerase promoter; and initiating RNA transcription using an RNA polymerase which recognizes the RNA polymerase promoter, thereby synthesizing at least one sRNA molecule. Applicants have found that such methods provide robust linear sRNA amplification with minimal non-specific amplification.

The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994).

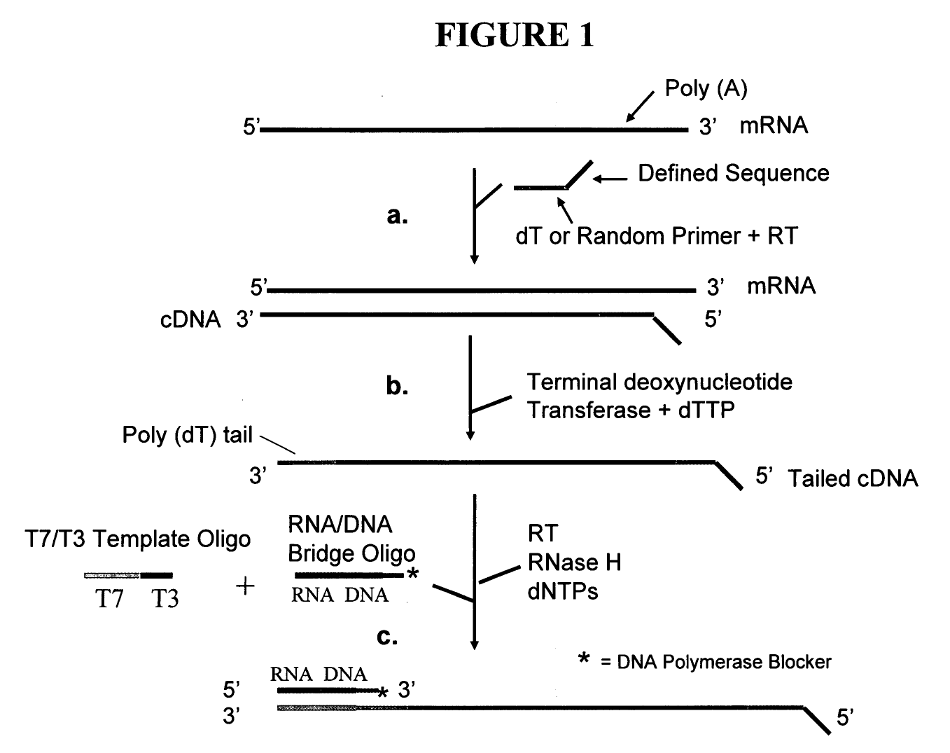

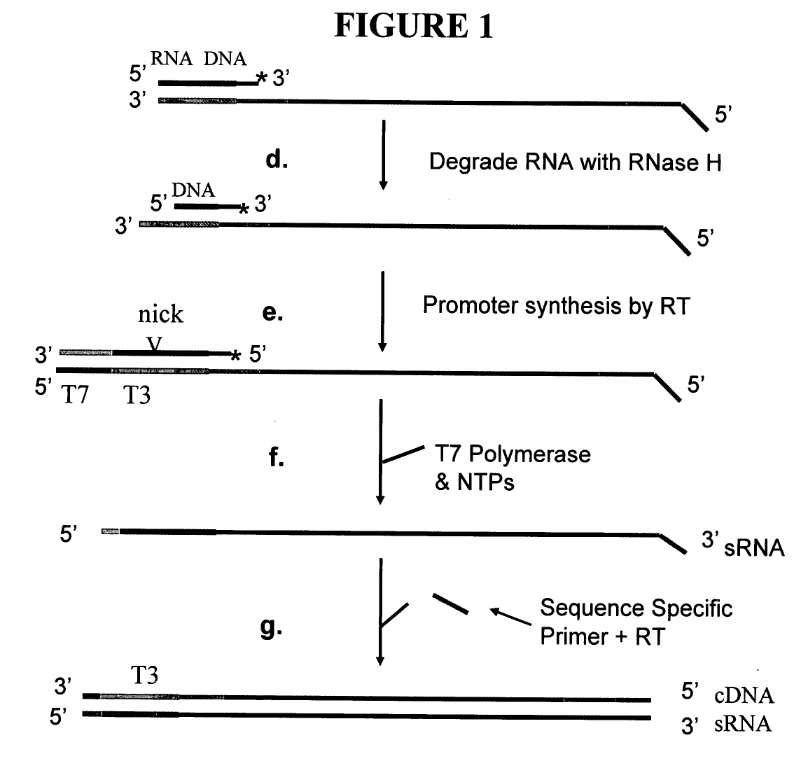

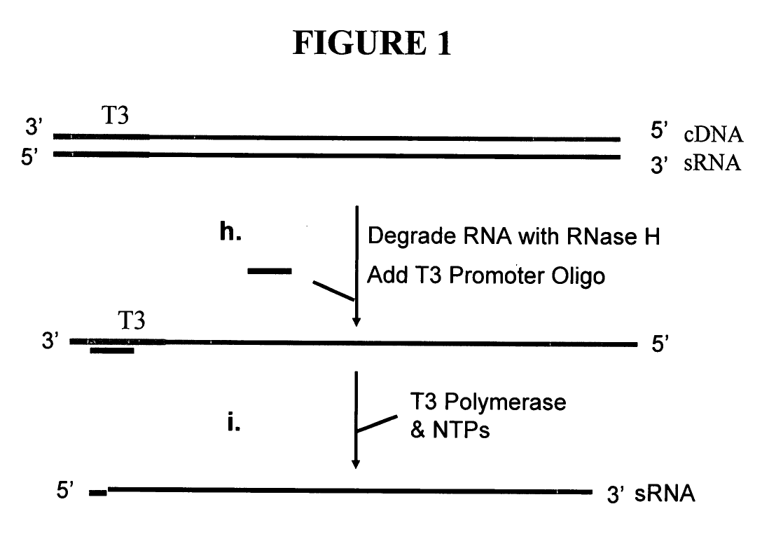

Numerous methods and commercial kits for the synthesis of first strand cDNA molecules are well known in the art. Examples include the Superscript™ Double Strand cDNA Synthesis Kit (Invitrogen, Carlsbad, Calif.), the Array 50™, Array 350™ and Array 900™ Detection Kits (Genisphere, Hatfield, Pa.), and the CyScribe™ Post-Labelling Kit (Amersham, Piscataway, N.J.). With reference to FIG. 1, RNA molecules (e.g., mRNA, hnRNA, rRNA, tRNA, mRNA, snoRNA, non-coding RNAs) from a source of interest are used as templates in a reverse transcription reaction (see FIG. 1a). The RNA may be obtained from any tissue or cell source, including virion, prokaryotic, and eukaryotic sources found in any biological or environmental sample. Preferably, the source is eukaryotic tissue, more preferably mammalian tissue, most preferably human tissue. The methods of present invention are particularly suited for amplification of RNA from small numbers of cells, including single cells, which can be purified from complex cellular samples using, e.g., micromanipulation, fluorescence-activated cell sorting (FACS) and laser microdissection techniques (see Player et al., Expert Rev. Mol. Diagn. 4:831 (2004)).

Any reverse transcriptase can be used in the initial reverse transcription reaction, including thermostable, RNAse H⁺ and RNase H⁻ reverse transcriptases. Preferably, an RNase H⁻ reverse trancriptase is used.

Primers for first strand cDNA synthesis can be obtained commercially or synthesized and purified using techniques well known in the art. Primers for first strand cDNA synthesis include single strand oligodeoxynucleotides comprising an oligodT tail at their 3′ ends, generally ranging from about 10 to about 30 nucleotides in length, preferably from about 17 to about 24 nucleotides in length, which anneal to RNA containing a 3′ polyA tail (e.g., mRNA). If the RNA of interest does not naturally contain a 3′ polyA tail (e.g., mRNA), a polyA tail can be attached to the RNA molecules using polyA polymerase (PAP) in the presence of ATP. PolyA tailing kits are commercially available and include, e.g., the Poly(A) Tailing Kit (Ambion, Austin, Tex.). Three-prime blocked RNAs can be enzymatically treated to allow tailing using, e.g., calf intestinal alkaline phosphatase or RNase 3.

Alternatively, the reverse transcription reaction can be initiated using a random primer, generally ranging from about 4 to about 20 nucleotides in length, preferably from about 6 to about 9 nucleotides in length, which anneals to various positions along the length of each original mRNA transcript. One of ordinary skill in the art will recognize that the use of a random primer can ultimately result in the production of sRNA molecules that are better representative of the entire length of each original mRNA transcript than those produced using an oligodT primer. Additionally, the use of a random primer to generate cDNA in the initial steps of the disclosed methods means that RNA that would normally be exempt from amplification, such as degraded RNA or RNA derived from bacteria, can be used to produce amplified sRNA molecules.

In some embodiments, the reverse transcription primer (oligodT primer, random primer, or both) comprises a 5′ extension containing a specific nucleotide sequence, generally ranging from about 6 to about 50 nucleotides in length, preferably from about 10 to about 20 nucleotides in length. This 5′ specific nucleotide sequence can be used as an initiation site for second round cDNA synthesis (see FIG. 1g).

In other embodiments, the 3′ terminal nucleotide of the reverse transcription primer (oligodT primer, random primer, or both) is a nucleotide or nucleotide analog that is not a substrate for terminal deoxynucleotide transferase but can be extended by reverse transcriptase, such as a ribonucleotide. Such primers are not extendable with terminal deoxynucleotidyl transferase (TdT), and thus will not be tailed and amplified in the steps shown in FIGS. 1b-1f. In preferred embodiments, the reverse transcription primer comprises a 5′ extension containing a specific nucleotide sequence, wherein the 3′ terminal nucleotide of the reverse transcription primer is a ribonucleotide.

Following first strand cDNA synthesis, the resulting first round cDNA molecules are generally purified (see FIG. 1b). While not degrading the RNA prior to cDNA purification is preferred, cDNA that has been purified following RNA degradation works equally well in the methods of the present invention. Any method that degrades RNA can be used, such as treatment with NaOH or RNase H (whether supplied in the form of a RNase H⁺ reverse transcriptase or as a separate enzyme). Alternatively, the RNA can be left intact, with the first round cDNA molecules purified from RNA/cDNA duplexes. Numerous methods and kits exist for the purification of DNA molecules, including, e.g., the MinElute™ PCR Purification Kit (Qiagen, Valencia, Calif.). If a reverse transcription primer is used for first strand cDNA synthesis in which the 3′ terminal nucleotide is a ribonucleotide, DNA purification can be omitted. This may reduce sample loss and increase amplification yield, which is particularly important when manipulating RNA from small numbers of cells.

Following first round cDNA purification, a single stranded oligodeoxynucleotide tail is generally attached to the 3′ end of the cDNA molecules (see FIG. 1b). The use of such oligodeoxynucleotide tails allows whole populations of nucleic acid molecules to be amplified, rather than just specific sequences. The oligodeoxynucleotide tail can be incorporated by any means that attaches deoxynucleotides to DNA. Preferably, the oligodeoxynucleotide tail is attached to the cDNA using terminal deoxynucleotidyl transferase, or other suitable enzyme, in the presence of appropriate deoxynucleotides. Preferably, the oligodeoxynucleotide tail is a homopolymeric tail (i.e., polydA, polydG, polydC, or polydT). Preferably, the oligodeoxynucleotide tail is a polydA tail, generally ranging from about 3 to greater than 500 nucleotides in length, preferably from about 20 to about 100 nucleotides in length. Applicants have found that the use of a polydA tail reduces the number of artifacts resulting from non-specific amplification.

Following attachment of the single stranded oligonucleotide tail to the 3′ ends of the cDNA molecules, a single stranded RNA/DNA composite bridge oligonucleotide comprising a 5′ RNA portion and a 3′ DNA portion is annealed to the 3′ oligodeoxynucleotide tail (see FIG. 1c). This is accomplished through complementary base pairing between the 3′ oligodeoxynucleotide tail and at least a portion of the 3′ DNA portion of the RNA/DNA composite bridge oligonucleotide. For example, if oligonucleotide tail is a polydT tail, the 3′ DNA portion of the RNA/DNA composite bridge oligonucleotide will contain a series of adenines at its 3′ end, generally ranging from about 3 to greater than 50 nucleotides in length, preferably from about 10 to about 30 nucleotides in length. The particular deoxynucleotide sequence of the 3′ DNA portion of the RNA/DNA composite bridge oligonucleotide does not have to be perfectly complementary to the particular nucleotide sequence of the oligodeoxynucleotide tail at the 3′ ends of the cDNA molecules, nor do their lengths need to match exactly, for the sequences to be considered complementary to each other. Those of skill in the art will recognize that what is required is that there be sufficient complementarity between the two sequences so that the RNA/DNA composite bridge oligonucleotide can anneal to the oligodeoxynucleotide tail at the 3′ end of the cDNA molecules.

In some embodiments, rather than attaching a single stranded oligodeoxynucleotide tail to the 3′ ends of the cDNA molecules, a single stranded RNA/DNA composite bridge oligonucleotide in which the DNA portion comprises random nucleotides is annealed to the cDNA molecules. Again, the use of such a random composite bridge oligonucleotide allows whole populations of nucleic acid molecules to be amplified, rather than just specific sequences. The random DNA portion of the composite oligonucleotide generally ranges from about 3 to greater than 50 nucleotides in length, preferably from about 6 to about 20 nucleotides in length. Only those bridge oligonucleotides that hybridize to the 3′ ends of the cDNA molecules will result in the synthesis of functional RNA polymerase promoters as described below. Hybridization is preferably performed at about 37° C. to about 55° C., more preferably at 45° C. to about 50° C.

In addition to the 3′ DNA portion (whether random or defined), the composite bridge oligonucleotide contains a 5′ RNA portion which remains single stranded (i.e., unannealed) following the annealing of the 3′ DNA portion of the composite bridge oligonucleotide to the 3′ oligodeoxynucleotide tail. The 5′ RNA portion generally ranges from about 3 to greater than 50 nucleotides in length, preferably from about 10 to about 30 nucleotides in length. Preferably, the particular sequence of the 5′ RNA portion is not substantially homologous to any known nucleic acid sequence, nor is it substantially self-complementary or complementary to any portion of the single stranded RNA polymerase promoter template described below.

The RNA/DNA composite bridge oligonucleotide can be blocked at its 3′ end if desired, such that it is not extendable with a DNA polymerase. As such, the addition of reverse transcriptase with both RNA-dependent and DNA-dependent DNA polymerase activity (e.g., MMLV reverse transcriptase, AMV reverse transcriptase, RBst DNA polymerase (Epicentre Technologies, Madison, Wis.)) and dNTPs extends the single stranded 3′ oligonucleotide tail at the 3′ ends of the cDNA molecules such that the RNA portion of the bridge oligonucleotide becomes a double stranded RNA/DNA duplex, but does not catalyze the synthesis of second strand cDNA (see FIG. 1c). The RNA/DNA composite bridge oligonucleotide can be blocked by any means that renders it incapable of being extended with DNA polymerase, such as by including terminal blocking groups, compounds, or moieties either attached during or after synthesis. Preferably, the RNA/DNA composite bridge oligonucleotide is blocked with a 3′ amino modifier, a 3′ deoxyterminator, or a 3′ dideoxyterminator. A suitable blocker should not be restricted to any of those described herein and can include any moiety that will prevent a DNA polymerase from extending the 3′ terminus of the RNA/DNA composite bridge oligonucleotide.

Following extension of the 3′ oligonucleotide tail to form a RNA/DNA duplex, the RNA portion of the duplex (i.e., the RNA portion of the bridge oligonucleotide) is degraded with RNase to expose a 3′ single stranded DNA tail on the cDNA molecules (see FIG. 1d). Preferably, the RNase is RNase H, although other RNases, such as RNase 1 and RNase A can be used. The RNase can be provided as part of the reverse transcriptase or as a separate enzyme. The RNase is preferably added at substantially the same time as the reverse transcriptase and the bridge oligonucleotide (see FIG. 1c).

Following degradation of RNA portion of the RNA/DNA duplex, a single stranded RNA polymerase promoter template is attached to the exposed 3′ single stranded DNA tail on the cDNA molecules (see FIG. 1e). This is accomplished through complementary base pairing between the exposed 3′ single stranded DNA tail and a complementary series of nucleotides present at the 3′ end of the single stranded promoter template, generally ranging from about 3 to greater than 50 nucleotides in length, preferably from about 10 to about 30 nucleotides in length.

The single stranded promoter template contains at its 5′ end at least one RNA polymerase recognition sequence. The promoter template can be composed of RNA and/or DNA, and can be blocked or unblocked at its 3′ end. When composed of both RNA and DNA, the 3′ portion of the promoter template that hybridizes to the exposed DNA tail on the cDNA molecules is preferably DNA, while the 5′ unhybridized portion is RNA. For performing multiple rounds of sRNA synthesis, the promoter template preferably contains at least a second different RNA polymerase recognition sequence 3′ to the first recognition sequence (i.e., a “tandem promoter template”; see FIG. 1c) (see Applicants' co-pending U.S. patent application Ser. No. 11/150,794, specifically incorporated herein by reference in its entirety). The term “RNA polymerase recognition sequence” is intended to cover both single stranded and double stranded nucleotide sequences. When in single stranded form, the nucleotide sequence corresponds to the non-template strand of a double-stranded RNA polymerase promoter. When in double stranded form, the nucleotide sequences correspond to both the template and non-template strands of a double-stranded RNA polymerase promoter. Any RNA polymerase recognition sequence can be used, so long as it is specifically recognized by an RNA polymerase. Preferably, the RNA polymerase recognition sequence(s) used is recognized by a bacteriophage RNA polymerase, such as T7, T3, or SP6 RNA polymerase. An exemplary T7 RNA polymerase recognition sequence is TAATACGACTCACTATAGGG (SEQ ID NO: 1). An exemplary T3 RNA polymerase recognition sequence is AATTAACCCTCACTAAAGGG (SEQ ID NO: 2). An exemplary SP6 RNA polymerase recognition sequence is AATTTAAGGTGACACTATAGAA (SEQ ID NO: 3). The RNA polymerase promoter template is preferably added at substantially the same time as the reverse transcriptase, bridge oligonucleotide and RNase (e.g., in the same reaction vessel) (see FIG. 1c), although each of the reactions can be performed separately.

Following attachment, the reverse transcriptase from FIG. 1c, having DNA-dependant DNA polymerase activity, extends the exposed 3′ single stranded DNA tail on the cDNA molecules and converts the single stranded promoter template into a double stranded RNA polymerase promoter (see FIG. 1e). Critically, even unblocked promoter templates are not extended during the reaction because reverse transcriptase lacks 5′→3′ exonuclease and strand displacement activities. Alternatively, or in addition, to reverse transcriptase, a DNA polymerase, such as T4 DNA polymerase, T7 DNA polymerase, or Sequenase™ (USB Corporation, Cleveland, Ohio), all of which lack 5′→3′ exonuclease and strand displacement activities, can be used to extend the exposed 3′ single stranded DNA tail on the cDNA molecules and convert the single stranded promoter template into a double stranded RNA polymerase promoter. Klenow enzyme has even been shown in the present system to convert the promoter template into a RNA polymerase promoter without extending the template when added near the end of the reverse transcriptase/RNase promoter synthesis reaction(s) (e.g., about 5 min to about 15 min before the completion of promoter synthesis). The use of such DNA polymerases may prevent or correct incorporation errors associated with the use of reverse transcriptase alone.

To further ensure that unblocked promoter templates are not extended during the promoter synthesis reaction(s), a nucleotide extension can be included at the 3′ end of an unblocked single stranded promoter template. This 3′ terminal nucleotide extension, downstream of the complementary 3′ series of deoxynucleotides used to attach the promoter template to the exposed 3′ single stranded DNA tail on the cDNA molecules, comprises a series of nucleotides identical to the 5′ end of the remaining DNA portion of bridge oligonucleotide, generally ranging from about 3 to about 10 nucleotides in length. As such, the 3′ extension, which would bind to the cDNA molecules but for the presence of the remaining DNA portion of bridge oligonucleotide, functions to prevent access to the gap or nick present between the promoter template and the remaining DNA portion of the bridge oligonucleotide during promoter synthesis (see FIG. 1e). Thus, any potential strand displacement during promoter synthesis is prevented as long as a DNA polymerase incapable of degrading the 3′ nucleotide extension is used in the synthesis reactions(s) (e.g., Klenow exo⁻).

In some embodiments, rather than enzymatically synthesizing a double stranded RNA polymerase promoter from a single stranded promoter template, a double stranded RNA polymerase promoter having a template strand and a non-template strand is attached to the 3′ ends of the first round cDNA molecules by DNA ligation (see Applicant's co-pending International Patent Application No. PCT/US2004/014325, specifically incorporated herein by reference in its entirety). The double stranded RNA polymerase promoter contains at its 5′ end (relative to the non-template strand) at least one RNA polymerase recognition sequence. For performing multiple rounds of sRNA synthesis, the double stranded RNA polymerase promoter preferably contains at least a second different RNA polymerase recognition sequence 3′ to the first recognition sequence (i.e., a “tandem promoter template”) (see Applicants' co-pending U.S. patent application Ser. No. 11/150,794, specifically incorporated herein by reference in its entirety). Attachment of the promoter is facilitated by complementary base pairing between the exposed 3′ single stranded DNA tail on the cDNA molecules and an overhang sequence at the 3′ end of the non-template strand of the double stranded RNA polymerase promoter that contains a complementary series of nucleotides, generally ranging from about 3 to greater than 50 nucleotides in length, preferably from about 10 to about 30 nucleotides in length. Once properly positioned, the double stranded promoter is attached to the cDNA molecule by ligation of the 5′ end of the template strand of the promoter to the 3′ end of the exposed single stranded DNA tail. Any DNA ligase can be used in the ligation reaction. Preferably, the DNA ligase is T4 DNA ligase.

Although the methods of current invention are preferably performed in the absence of second strand cDNA synthesis, one of skill in the art will recognize that second strand cDNA can be optionally synthesized during conversion of the single stranded promoter template into a double stranded RNA polymerase promoter by using a random primer. The random primer will anneal at various positions along the first strand cDNA and be extended by DNA-dependant DNA polymerase activity of reverse transcriptase during promoter synthesis. The various second strand cDNA fragments can be optionally ligated together to form a single second strand cDNA molecule. Such second strand cDNA molecules may stabilize (i.e., remove secondary and tertiary structure) the first strand cDNA during in vitro transcription, resulting in a higher yield of sRNA molecules.

Following conversion of the single stranded promoter template into a double stranded RNA polymerase promoter, in vitro transcription is initiated by the addition of ribonucleotides and a RNA polymerase that recognizes the promoter (see FIG. 1f). If a tandem promoter template was attached to the cDNA molecules (see FIG. 1e), in vitro transcription is preferably initiated using a RNA polymerase that recognizes the first 5′ promoter (see FIG. 1f). This facilitates second round sRNA synthesis described in further detail below. Methods and kits for performing in vitro transcription are well known in the art and include the MEGAscript™ Transcription Kit (Ambion) and the AmpliScribe™ High Yield Transcription Kits (Epicentre Technologies).

Additional rounds of sRNA synthesis can be performed by reverse transcribing the resulting first round sRNA molecules (i.e., second round cDNA molecules) and re-attaching a promoter template onto the second-round cDNA molecules as just described, followed by enzymatic conversion to the double stranded promoter and a second round of in vitro transcription with RNA polymerase. If, however, a tandem promoter template was attached to the first round cDNA molecules (see FIG. 1e), and in vitro transcription initiated using a RNA polymerase that recognizes the first 5′ promoter (see FIG. 1f), additional rounds of sRNA synthesis can be performed without the need for re-attachment of a promoter template and re-synthesis of the double stranded promoter (see Applicants' co-pending U.S. patent application Ser. No. 11/150,794, specifically incorporated herein by reference in its entirety).

The first round sRNA molecules are first subjected to a second round of synthesis by first reverse transcribing the sRNA molecules into first strand cDNA molecules as described above (see FIG. 1g). For example, sRNA molecules produced from oligodT-primed first strand cDNA will have regenerated polyA tails at their 3′ ends, which can serve as priming sites for a second round of oligodT-primed first strand cDNA synthesis. Additionally, and for first round sRNA molecules produced from random-primed first strand cDNA, 3′ polyA tails can be added to the sRNA molecules for oligodT-primed first strand cDNA synthesis, or random primer-mediated reverse transcription can again be performed to produce second round cDNA. Combinations and mixtures of oligodT and random primers can also be used for second round cDNA synthesis.

If the first round reverse transcription primer used in the step shown in FIG. 1a comprises a 5′ extension containing a specific nucleotide sequence, the first round sRNA molecules will contain a defined complementary nucleotide sequence at their 3′ ends. Reverse transcription can be initiated using a second reverse transcription primer comprising a nucleotide sequence complementary to this defined nucleotide sequence (i.e., “corresponding” to the specific nucleotide sequence of the 5′ extension) (see FIG. 1g). Only first round sRNA molecules containing the defined nucleotide sequence will be reversed transcribed, resulting in reduced non-specific amplification. Alternatively, second round reverse transcription in this embodiment can be initiated using the oligodT primer and/or random primer used for first round cDNA synthesis (or another suitable primer).

Following second round cDNA synthesis, the RNA strand is degraded using NaOH or preferably RNase H prior to optional purification of the first strand cDNA molecules (see FIG. 1h). Similarly, an RNase H⁺ reverse transcriptase can be used, such as MMLV.

Following RNA degradation, a single stranded promoter oligonucleotide complementary to the second different 3′ RNA polymerase recognition sequence is annealed to the second round cDNA molecules through complementary base pairing (see FIG. 1h). This base pairing forms a second RNA polymerase promoter, from which a second round of in vitro transcription (i.e., second round sRNA molecules) is initiated by the addition of ribonucleotides and a RNA polymerase that recognizes the second promoter (see FIG. 1i). By incorporating additional different RNA polymerase recognition sequences into the promoter template, additional rounds of sRNA synthesis can be performed as described (e.g., third round sRNA molecules, etc.). Further, by heat inactivating all enzymes between steps or before addition of RNA polymerase, using methods familiar to one skilled in the art, linear, rather than exponential, amplification can be maintained. Such linear amplification is better suited for various downstream applications, such as gene expression studies. It should be understood that unless otherwise specified, all enzyme activity is terminated either before the next enzymatic manipulation or prior to adding RNA polymerase.

In some embodiments, rather than inactivating the reverse transcriptase following second round cDNA synthesis and annealing a single stranded promoter oligonucleotide complementary to the second different RNA polymerase recognition sequence, the RNA strand is degraded using Rnase H and the tandem promoter is regenerated by the binding of excess single stranded tandem promoter template (from the first round) to the 3′ ends of the second round cDNA molecules and the DNA-dependent DNA polymerase activity of the still-active reverse transcriptase (see Applicants' co-pending U.S. patent application Ser. No. 11/150,794, specifically incorporated herein by reference in its entirety). A second round of in vitro transcription can then be initiated by the addition of an RNA polymerase that recognizes either the first or second promoter. Again, the reverse transcriptase is generally heat inactivated just prior to addition of RNA polymerase to maintain the linearity of the amplification. Those of skill in the art will recognize that the single stranded promoter template in these embodiments need not contain two RNA polymerase recognition sequences in tandem. Rather, the promoter template can contain a single RNA polymerase recognition sequence, which can be used in place of the tandem promoter template to produce first and second round sRNA molecules.

The sRNA molecules produced by the methods of the present invention can be used directly for any purpose mRNA is typically used for, including gene expression studies, genetic cloning, subtractive hybridization, and other techniques familiar to one experienced in the art. Preferably, the sRNA molecules are reverse transcribed into cDNA molecules using random primers, oligodT primers, or combinations thereof. The reverse transcription reaction can be performed directly in the presence of detectably labeled nucleotides, such as fluorescently labeled nucleotides. Such nucleotides include nucleotides labeled with Cy3 and Cy5.

Alternatively, the cDNA molecules are labeled indirectly. For example, the reverse transcription reaction can be performed in the presence of biotinylated or amino allyl nucleotides (e.g., amino allyl UTP), followed by coupling to a NHS ester label (e.g., Cy dye). Preferably, the cDNA molecules are labeled indirectly using 3DNA™ dendrimer technology (Genisphere, Hatfield, Pa.). Dendritic reagents are further described in Nilsen et al., J. Theor. Biol., 187:273 (1997); in Stears et al., Physiol. Genomics, 3:93 (2000); and in various U.S. patents, such as U.S. Pat. Nos. 5,175,270; 5,484,904; 5,487,973; 6,072,043; 6,110,687; and 6,117,631, each specifically incorporated herein by reference in its entirety.

The sRNA molecules can also be used in cRNA amplification procedures to produce labeled antisense RNA (asRNA) molecules. For example, using the method of Eberwine et al. (see, e.g., Van Gelder et al., Proc. Natl. Acad. Sci. USA 87:1663 (1990); U.S. Pat. Nos. 5,545,522; 5,716,785; 5,891,636; 5,958,688; and 6,291,170, each specifically incorporated herein by reference in its entirety), a T7 promoter primer can be used to reverse transcribe the sRNA molecules. Following second strand cDNA synthesis, RNA transcription is initiated using T7 RNA polymerase, producing amplified asRNA molecules. Such asRNA molecules can be labeled directly during synthesis by incorporating labeled nucleotides (e.g., Cy-labeled nucleotides), or can be indirectly labeled by e.g., incorporating a biotinylated or amino allyl nucleotide (e.g., amino allyl UTP), followed by coupling to a NHS ester label (e.g., Cy dye).

The labeled single stranded cDNA and asRNA molecules produced from the sRNA molecules of the present invention are useful as reagents for gene expression studies. The labeled cDNA and asRNA molecules can be annealed to a nucleic acid microarray containing complementary polynucleotides (e.g., probes). As used herein, “microarray” is intended to include any solid support containing nucleic acid probes, including slides, chips, membranes, beads, and microtiter plates. Examples of commercially available microarrays include the GeneChip® microarray (Affymetrix, Santa Clara, Calif.), CodeLink™ microarray (Amersham Biosciences, Piscataway, N.J.), Agilent (Palo Alto, Calif.) Oligo microarray, and OciChip™ microarray (Ocimum Biosolutions, Indianapolis, Ind.).

The methods and compositions of the present invention can be conveniently packaged in kit form. Such kits can be used in various research and diagnostic applications. For example, methods and kits of the present invention can be used to facilitate a comparative analysis of expression of one or more genes in different cells or tissues, different subpopulations of the same cells or tissues, different physiological states of the same cells or tissue, different developmental stages of the same cells or tissue, or different cell populations of the same tissue. Such analyses can reveal statistically significant differences in the levels of gene expression, which, depending on the cells or tissues analyzed, can then be used to facilitate diagnosis of various disease states.

A wide variety of kits may be prepared according to present invention. For example, a kit may include a single stranded promoter template comprising at least one RNA polymerase recognition sequence; a single stranded RNA/DNA composite bridge oligonucleotide comprising a RNA sequence 5′ of a DNA sequence; and instructional materials for synthesizing sRNA molecules using said promoter template and said RNA/DNA composite bridge oligonucleotide. For performing additional rounds of sRNA synthesis, the kit can further include a single stranded promoter oligonucleotide complementary to a second RNA polymerase recognition sequence of the promoter template and the appropriate instructional materials. While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media may include addresses to internet sites that provide such instructional materials.

The kits of the present invention may further include one or more of the following components or reagents: a reverse transcriptase (preferably with DNA-dependent DNA polymerase activity); an RNase inhibitor; an enzyme for attaching a 3′ oligodeoxynucleotide tail onto DNA molecules (e.g., terminal deoxynucleotidyl transferase); an enzyme for degrading RNA in RNA/DNA duplexes (e.g., RNase H); and one or more RNA polymerases (e.g., T7, T3 or SP6 RNA polymerase). Additionally, the kits may include buffers, primers (e.g., oligodT primers, random primers), nucleotides, labeled nucleotides, an RNase inhibitor, polyA polymerase, RNase-free water, containers, vials, reaction tubes, and the like compatible with the synthesis of sRNA molecules according to the methods of the present invention. The components and reagents may be provided in containers with suitable storage media.

Specific embodiments according to the methods of the present invention will now be described in the following examples. The examples are illustrative only, and are not intended to limit the remainder of the disclosure in any way.

### EXAMPLES

### Example 1

One Round of sRNA Synthesis

1. First Strand cDNA Synthesis

For each RNA sample, purified using the RNAqueous® Kit (Ambion), the following RNA/primer mix was prepared on ice:
1-8 μl total RNA (not exceeding 2 ng)
2 μl oligodT sequence specific RT primer (50 ng/μl) (5′-TAC AAG GCA ATT TTT TTT TTT TTT TTT V-3′, where V=C, G or A deoxyribonucleotides; SEQ ID NO: 4)
1 μl random sequence specific RT primer (2× by mass of RNA) (5′-TAC AAG GCA ATT NNN NNN NNN-3, where N=A, G, C or T deoxyribonucleotides at random; SEQ ID NO: 5)
RNase-free water to 11 μl

The RNA/primer mixture was heated at 80° C. for 10 minutes and immediately cooled on ice for 1-2 min. The mixture was then mixed with 9 μl of a Master Mixture solution to bring the final volume to 20 μl containing 1×RT buffer (50 mM Tris-HCl (pH 8.3), 75 mM KCl, 3 mM MgCl₂), 10 mM dithiothreitol (DTT), 0.5 mM each dNTP, 10 U Superase-In™ (Ambion), and 200 U Superscript™ II reverse transcriptase (Invitrogen). The mixture was briefly centrifuged and incubated at 42° C. for 2 hrs. Following a brief centrifugation, the reaction was adjusted to 100 μl with 1×TE (10 mM Tris-HCl, pH 8.0, 1 mM EDTA).

2. cDNA Purification

The reaction was purified using the MinElute™ PCR Purification Kit (Qiagen) according to the manufacturer's protocol. Briefly, the cDNA reaction was adjusted to 600 μl with PB buffer provided by the manufacturer. The cDNA reaction was applied to the MinElute™ column and microfuged for 1 minute. The flow-through in the collection tube was discarded, and the column washed with 750 μl PE buffer provided by the manufacturer. The flow-through in the collection tube was discarded, and the column washed with 500 μl 80% ethanol. The flow-through in the collection tube was discarded, and the column microfuged with the cap open for 5 minutes to dry the resin. The column was placed in a clean 1.5 ml microfuge tube, and the column membrane incubated with 10 μl EB buffer provided by the manufacturer for 2 minutes at room temperature. The first strand cDNA molecules were eluted by microfugation for 2 minutes.

3. Tailing of First Strand cDNA

The first strand cDNA molecules were heated at 80° C. for 10 minutes and immediately cooled on ice for 1-2 min. The cDNA molecules in 10 μl were then mixed with 10 μl of a Master Mixture solution to bring the final volume to 20 μl containing 1× Tailing buffer (10 mM Tris-HCl, pH 7.0, 10 mM MgCl₂), 0.04 mM dTTP, and 15 U terminal deoxynucleotidyl transferase (Roche Diagnostics, Indianapolis, Ind.). The mixture was briefly centrifuged and incubated at 37° C. for 2 min. The reaction was stopped by heating at 80° C. for 10 min and cooled at room temperature for 1-2 minutes.

4. T7 Promoter Synthesis

One μl of T7 RNA polymerase promoter template oligonucleotide (5′-CAC TAA TAC GAC TCA CTA TAG GGA GAA ATT-3′; SEQ ID NO: 6) (100 ng/μl) and 1 μl of RNA/DNA composite bridge oligonucleotide (5′-rUrArG rGrGrA rGrArA rArUrU CGA CAC AAA AAA AAA AAA AAA-3′; SEQ ID NO: 7) (100 ng/μl) containing a 3′ amino modifier was added to the oligodT-tailed cDNA molecules and the mixture incubated at 37° C. for 10 min to anneal the strands. The bridge oligonucleotide contains a 5′ portion composed of ribonucleotides upstream of a 3′ portion composed of deoxynucleotides. The 3′ deoxynucleotide portion is designed to anneal to the 3′ ends of the polydT-tailed cDNA molecules. The 5′ ribonucleotide portion of the bridge oligonucleotide is degraded by RNase H once the cDNA molecules are extended by the DNA polymerase activity of MMLV reverse transcriptase, exposing 3′ single stranded tails on the cDNA molecules to which the 3′ end of the T7 RNA polymerase promoter template oligonucleotide is designed to anneal (see FIGS. 1c-1e). The 3′ amino modifier prevents extension of the bridge oligonucleotide during the promoter synthesis reaction, while MMLV reverse transcriptase's lack of 5′→3′ exonuclease and strand displacement activities ensure that the T7 RNA polymerase promoter template oligonucleotide is also not extended during the reaction.

The tailed cDNA molecules/bridge oligonucleotide/promoter template mixture was then mixed with 3 μl of a Master Mixture solution to bring the final volume to 25 μl containing 1× Polymerase buffer (10 mM Tris-HCl, pH 7.0, 10 mM MgCl₂), 0.4 mM each dNTP, 200 U Superscript II reverse transcriptase (Invitrogen) and 2 U RNase H (Invitrogen). The mixture was briefly centrifuged and incubated at 37° C. for 30 minutes. The reaction was stopped by heating at 65° C. for 15 min and placed on ice.

5. T7 In Vitro Transcription

One-half of the promoter synthesis reaction (12.5 μl) was heated at 37° C. for 10-15 min to re-anneal the T7 promoter strands and then mixed with 12.5 μl of a Master Mixture solution to bring the final volume to 25 μl containing 1× Reaction buffer, 7.5 mM each rNTP, and 2 μl T7 RNA polymerase (MEGAscript™ Transcription Kit, Ambion). The mixture was briefly centrifuged and incubated in a thermocycler with a heated lid at 37° C. for 4-16 hrs. Alternatively, the mixture was incubated in a 37° C. heat block for 15 min, followed by incubation in an air hybridization oven at 37° for 4-16 hrs. It is essential to avoid evaporation and condensation of the reaction during this step.

6. sRNA Purification and Quantitation

The sRNA molecules were purified using the RNeasy Kit (Qiagen) following manufacturer's protocol for RNA cleanup. The purified sRNA molecules were eluted twice in 50 μl RNase-free water and quantified by UV-spectrophotometry in 0.1×TE Buffer, pH 8.0 at a wavelength ratio of 260/280.

Replicate amplifications were performed starting with 1 μg of total RNA or water alone (negative control). On average, 50-75 μg of amplified sRNA was recovered after amplifying 1 μg of total RNA vs. less than 0.5 μg of non-specific amplification product when using only water in the reverse transcription reaction in place of RNA.

### Example 2

Two Rounds of sRNA Synthesis

1. First Strand cDNA Synthesis

For each RNA sample, purified using the RNAqueous® Kit (Ambion), the following RNA/primer mix was prepared on ice:
1-8 μl total RNA (not exceeding 2 ng)
2 μl first round oligodT sequence specific RT primer (50 ng/μl) (5′-TAC AAG GCA ATT TTT TTT TTT TTT TTT V-3′, where V=C, G or A deoxyribonucleotides; SEQ ID NO: 4)
1 μl first round random sequence specific RT primer (2× by mass of RNA) (5′-TAC AAG GCA ATT NNN NNN NNN-3, where N=A, G, C or T deoxyribonucleotides at random; SEQ ID NO: 5)
RNase-free water to 11 μl

The first round RT primers comprise a 5′ extension containing a specific nucleotide sequence that serves as binding sites for second round RT primers (see FIG. 1g). The RNA/primer mixture was heated at 80° C. for 10 minutes and immediately cooled on ice for 1-2 min. The mixture was then mixed with 9 μl of a Master Mixture solution to bring the final volume to 20 μl containing 1×RT buffer (50 mM Tris-HCl (pH 8.3), 75 mM KCl, 3 mM MgCl₂), 10 mM dithiothreitol (DTT), 0.5 mM each dNTP, 10 U Superase-In™ (Ambion), and 200 U Superscript™ II reverse transcriptase (Invitrogen). The mixture was briefly centrifuged and incubated at 42° C. for 2 hrs. Following a brief centrifugation, the reaction was adjusted to 100 μl with 1×TE (10 mM Tris-HCl, pH 8.0, 1 mM EDTA).

2. cDNA Purification

The reaction was purified using the MinElute™ PCR Purification Kit (Qiagen) according to the manufacturer's protocol. Briefly, the cDNA reaction was adjusted to 600 μl with PB buffer provided by the manufacturer. The cDNA reaction was applied to the MinElute™ column and microfuged for 1 minute. The flow-through in the collection tube was discarded, and the column washed with 750 μl PE buffer provided by the manufacturer. The flow-through in the collection tube was discarded, and the column washed with 500 μl 80% ethanol. The flow-through in the collection tube was discarded, and the column microfuged with the cap open for minutes to dry the resin. The column was placed in a clean 1.5 ml microfuge tube, and the column membrane incubated with 10 μl EB buffer provided by the manufacturer for 2 minutes at room temperature. The first strand cDNA molecules were eluted by microfugation for 2 minutes.

3. Tailing of First Strand cDNA

The first strand cDNA molecules were heated at 80° C. for 10 minutes and immediately cooled on ice for 1-2 min. The cDNA molecules in 10 μl were then mixed with 10 μl of a Master Mixture solution to bring the final volume to 20 μl containing 1× Tailing buffer (10 mM Tris-HCl, pH 7.0, 10 mM MgCl₂), 0.04 mM dATP, and 15 U terminal deoxynucleotidyl transferase (Roche Diagnostics). The mixture was briefly centrifuged and incubated at 37° C. for 2 min. The reaction was stopped by heating at 80° C. for 10 min and cooled at room temperature for 1-2 minutes.

4. T7/T3 Promoter Synthesis

One μl of T7/T3 RNA polymerase promoter template oligonucleotide (5′-TAA TAC GAC TCA CTA TAG GGA GAA ATT AAC CCT CAC T-3′; SEQ ID NO: 8) (100 ng/μl) and 1 μl of RNA/DNA composite bridge oligonucleotide (5′-rGrArA rArUrU rArArC rCrCrU rCrArC rUAA AGG GAT TTT TTT TTT TTT T-3′; SEQ ID NO: 9) (100 ng/μl) containing a 3′ amino modifier was added to the oligodA-tailed cDNA molecules and the mixture incubated at 37° C. for 10 min to anneal the strands. The T7/T3 RNA polymerase promoter template contains a T7 RNA polymerase promoter template 5′ to a T3 RNA polymerase recognition sequences. The tailed cDNA molecules/bridge oligonucleotide/promoter template mixture was then mixed with 3 μl of a Master Mixture solution to bring the final volume to 25 μl containing 1× Polymerase buffer (10 mM Tris-HCl, pH 7.0, 10 mM MgCl₂), 0.4 mM each dNTP, 200 U Superscript II reverse transcriptase (Invitrogen) and 2 U RNase H (Invitrogen). The mixture was briefly centrifuged and incubated at 37° C. for 30 minutes. The reaction was stopped by heating at 65° C. for 15 min and placed on ice.

5. T7 In Vitro Transcription

One-half of the promoter synthesis reaction (12.5 μl) was heated at 37° C. for 10-15 min to re-anneal the T7T3 promoter strands and then mixed with 12.5 μl of a Master Mixture solution to bring the final volume to 25 μl containing 1× Reaction buffer, 7.5 mM each rNTP, and 2 μl T7 RNA polymerase (MEGAscript™ Transcription Kit, Ambion). The mixture was briefly centrifuged and incubated in a thermocycler with a heated lid at 37° C. for 4-16 hrs. Alternatively, the mixture was incubated in a 37° C. heat block for 15 min, followed by incubation in an air hybridization oven at 37° for 4-16 hrs. It is essential to avoid evaporation and condensation of the reaction during this step.

6. Reverse Transcription of First Round sRNA

Twenty-five μl of first round sRNA was mixed with 1 μl second round sequence specific RT primer (500 ng/μl) (5′-TAC AAG GCA ATT-3′; SEQ ID NO: 10) and heated at 80° C. for 10 min. The second round RT primer contains a nucleotide sequence corresponding to the specific nucleotide sequence of the 5′ extension of the first round RT primers (see FIG. 1g). The reaction was immediately iced for 2 min, briefly centrifuged, and returned to ice. One μl dNTP mix (10 mM each) and 1 μl Superscript™ II reverse transcriptase (200 U/μl; Invitrogen) was added, and the RT reaction incubated at 42° C. for 1 hr. One μl RNase H (2 U/μl) (Invitrogen) was added, and the reaction incubated at 37° C. for 20 min. The reaction was then incubated at 65° C. to stop enzyme activity.

7. T3 Promoter Formation

Two μl of T3 promoter oligonucleotide (50 ng/μl) (5′-GAA ATT AAC CCT CAC TAA AGG G-3′; SEQ ID NO: 11) was added to the second round cDNA reaction. The T3 oligonucleotide is complementary to the T3 RNA polymerase recognition sequence of the initial T7/T3 RNA polymerase promoter template. The reaction was incubated at 37° for 10 min to anneal the strands.

8. T3 In Vitro Transcription

The T3 promoter synthesis reaction was mixed with 19 μl of a Master Mixture solution to bring the final volume to 25 μl containing 1× Reaction buffer, 7.5 mM each rNTP, and 2 μl T3 RNA polymerase (MEGAscript™ Transcription Kit, Ambion). The mixture was briefly centrifuged and incubated in a thermocycler with a heated lid at 37° C. for 4-16 hrs. Alternatively, the mixture was incubated in a 37° C. heat block for 15 min, followed by incubation in an air hybridization oven at 37° for 4-16 hrs. It is essential to avoid evaporation and condensation of the reaction during this step.

9. sRNA Purification and Quantitation

The second round sRNA molecules were purified using the RNeasy Kit (Qiagen) following manufacturer's protocol for RNA cleanup. The purified sRNA molecules were eluted twice in 50 μl RNase-free water and quantified by UV-spectrophotometry in 0.1×TE Buffer, pH 8.0 at a wavelength ratio of 260/280.

Replicate amplifications were performed starting with 1 ng of total RNA or water alone (negative control). On average, 25 μg of amplified sRNA was recovered after amplifying 1 ng of total RNA vs. 0.5-4 μg of non-specific amplification product when using only water in the reverse transcription reaction in place of RNA.

### Example 3

Each RNA sample was amplified as described in Example 1, except that only a non-specific oligodT primer was used for first round cDNA synthesis. The following RNA/primer mix was prepared on ice:
1-8 μl total RNA (not exceeding 2 ng)
2 μl first round oligodT RT primer (50 ng/μl) (5′-TTT TTT TTT TTT TTT TTT V-3′, where V=C, G or A; SEQ ID NO: 12)
RNase-free water to 11 μl

Replicate amplifications were performed starting with 1 μg of total RNA or water alone (negative control). On average, 8-10 μg of amplified sRNA was recovered after amplifying 1 μg of total RNA vs. 0.2 μg of non-specific amplification product when using only water in the reverse transcription reaction in place of RNA.

### Example 4

Each RNA sample was amplified as described in Example 1, except that only a non-specific random primer was used for first round cDNA synthesis. The following RNA/primer mix was prepared on ice:
1-8 μl total RNA (not exceeding 2 ng)
1 μl first round random sequence specific RT primer (2× by mass of RNA) (5′-NNN NNN NNN-3′, where N=A, G, C or T at random; SEQ ID NO: 13)
RNase-free water to 11 μl

Replicate amplifications were performed starting with 1 μg of total RNA or water alone (negative control). On average, 30-35 μg of amplified sRNA was recovered after amplifying 1 μg of total RNA vs. 0.2 μg of non-specific amplification product when using only water in the reverse transcription reaction in place of RNA.

### Example 5

Each RNA sample was amplified as described in Example 1, except that only the oligo dT sequence specific primer was used for first round cDNA synthesis. The following RNA/primer mix was prepared on ice:
1-8 μl total RNA (not exceeding 2 ng)
2 μl first round oligodT RT primer (50 ng/μl) (5′-TAC AAG GCA ATT TTT TTT TTT TTT TTT V-3′, where V=C, G or A; SEQ ID NO: 4)
RNase-free water to 11 μl

Replicate amplifications were performed starting with 1 μg of total RNA or water alone (negative control). On average, 8-10 μg of amplified sRNA was recovered after amplifying 1 μg of total RNA vs. 0.5 μg of non-specific amplification product when using only water in the reverse transcription reaction in place of RNA.

### Example 6

Gel Analysis of T7 Promoter Synthesis

1. T7 Promoter Synthesis

One μl of a 3′ polydT test oligonucleotide (5′-TTC TCG TGT TCC GTT TGT ACT CTA AGG TGG ATT TTT TTT TTT TTT TTT-3′; SEQ ID NO: 14) (50 ng/μl) was combined with 1 μl T7 RNA polymerase promoter template oligonucleotide (5′-CAC TAA TAC GAC TCA CTA TAG GGA GAA ATT-3′; SEQ ID NO: 6)(100 ng/μl) and 1 μl RNA/DNA composite bridge oligonucleotide (5′-rUrArG rGrGrA rGrArA rArUrU CGA CAC AAA AAA AAA AAA AAA-3′; SEQ ID NO: 7) (100 ng/μl) containing a 31 amino modifier and the mixture incubated at 37° C. for 10 min to anneal the strands. The polydT test oligonucleotide/bridge oligonucleotide/promoter template mixture was then mixed with 22 μl of a Master Mixture solution to bring the final volume to 25 μl containing 1× Polymerase buffer (10 mM Tris-HCl, pH 7.0, 10 mM MgCl₂), 0.4 mM each dNTP, 200 U Superscript II reverse transcriptase (Invitrogen) and 2 U RNase H (Invitrogen). The mixture was briefly centrifuged and incubated at 37° C. for 30 minutes. The reaction was stopped by heating at 65° C. for 15 min and placed on ice. A replicate control mixture was prepared without including the RNase H.

2. Gel Electrophoresis

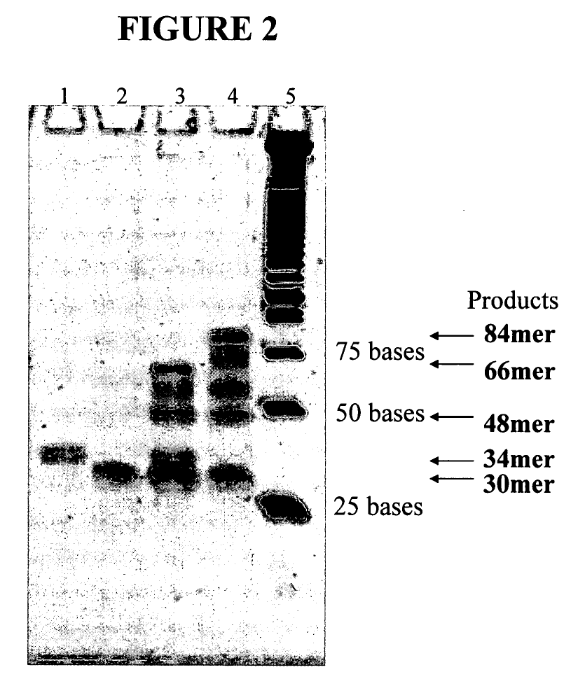

Five μl of Gel Loading Buffer (Ambion) was added to each of the samples and the samples loaded onto a Novex® 10% TBE Urea Gel (Invitrogen) along with a 25 bp ladder. The gel was run for 35 minutes at 65° C. and stained with SYBR® Gold Nucleic Acid Gel Stain (Invitrogen) for 30 minutes to visualize the bands (FIG. 2). As shown in lane 4, the combination of the 34mer RNA/DNA composite bridge oligonucleotide (lane 1), the 30mer T7 RNA polymerase promoter template oligonucleotide (lane 2) and the 48mer polydT test oligonucleotide in the presence of reverse transcriptase, dNTPs and RNase H yielded the predicted 84mer product, indicating that the T7 promoter was effectively synthesized by the process. When RNase H was not included in the reaction (lane 3), no T7 promoter was synthesized.

### Example 7

A kit for performing one or more rounds of sRNA synthesis was assembled with the following components:
First Round Oligo dT Sequence Specific RT Primer (50 ng/μl);
First Round Random Sequence Specific RT Primer (250 ng/μl);
dNTP Mix (10 mM each dATP, dCTP, dGTP, dTTP);
Superase-In™ RNase Inhibitor (Ambion);
10 mM dATP;
10× Reaction Buffer (100 mM Tris-HCl, pH 7.0, 100 mM MgCl₂);
Terminal Deoxynucleotidyl Transferase (7.5 U/μl);
RNA/DNA composite bridge oligonucleotide (100 ng/μl);
T7/T3 RNA Polymerase Promoter Template (50 ng/μl);
rNTP Mix (ATP, GTP, CTP, and UTP) (75 mM each);
10×RNA Polymerase Reaction Buffer (Ambion);
T7 Enzyme Mix (Ambion);
Second Round Sequence Specific RT Primer (500 ng/μl);
T3 Promoter Oligonucleotide (50 ng/μl);
T3 Enzyme Mix (Ambion);
RNase H (Invitrogen);
MMLV reverse transcriptase (Invitrogen); and
T4 DNA Polymerase (Invitrogen).

The components were placed in numbered vials and placed in a container with a printed instruction manual for multiple rounds of sRNA synthesis using the kit components.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A method for synthesizing at least one sRNA molecule, comprising:
(a) providing at least one single stranded cDNA molecule having a 5′ end and a 3′ end;
(b) attaching an oligodeoxynucleotide tail onto the 3′ end of said cDNA molecule;
(c) annealing to said oligodeoxynucleotide tail a single stranded RNA/DNA composite bridge oligonucleotide comprising a 5′ RNA portion and a 3′ DNA sequence portion, such that the RNA portion remains single stranded;
(d) extending the 3′ end of said oligodeoxynucleotide tail, such that said single stranded RNA portion becomes a double stranded RNA/DNA duplex;
(e) degrading the RNA portion of said RNA/DNA duplex, thereby exposing a 3′ single stranded DNA tail;
(f) annealing to said 3′ single stranded DNA tail a single stranded promoter template comprising at least one RNA polymerase recognition sequence;
(g) extending said 3′ single stranded DNA tail such that said at least one single stranded RNA polymerase promoter template is converted into at least one RNA polymerase promoter;
(h) and initiating RNA transcription using an RNA polymerase which recognizes said at least one RNA polymerase promoter,
thereby synthesizing at least one sRNA molecule.

2. The method of claim 1, wherein a) comprises providing at least one RNA molecule having 5′ and 3′ ends; and synthesizing at least one single stranded cDNA molecule from said at least one RNA molecule.

3. The method of claim 2, wherein synthesis of the single stranded cDNA molecule or molecules comprises contacting the RNA molecule or molecules with a primer in the presence of a reverse transcriptase.

4. The method of claim 3, wherein the primer is selected from the group consisting of oligodT primer, random primer, and combinations thereof.

5. The method of claim 4, wherein the primer comprises a 5′ extension containing a specific nucleotide sequence.

6. The method of claim 4, wherein the 3′ terminal nucleotide of the primer is not a substrate for terminal deoxynucleotide transferase but can be extended by reverse transcriptase.

7. The method of claim 6, wherein the 3′ terminal nucleotide of the primer is a ribonucleotide.

8. The method of claim 4, wherein the primer comprises a 5′ extension containing a specific nucleotide sequence, wherein the 3′ terminal nucleotide of said primer is a ribonucleotide.

9. The method of claim 1, wherein the single stranded RNA/DNA composite bridge oligonucleotide is blocked at its 3′ end such that it is not extendable with DNA polymerase.

10. The method of claim 1, wherein the RNA portion of the RNA/DNA duplex is degraded using Rnase H.

11. The method of claim 1, wherein the single stranded promoter template comprises a first RNA polymerase recognition sequence selected from the group consisting of T7, T3 and SP6 RNA polymerase recognition sequence and a second RNA polymerase recognition sequence selected from the group consisting of T7, T3 and SP6 RNA polymerase recognition sequence, wherein said first and second RNA polymerase recognition sequences are different.

12. The method of claim 1, wherein the single stranded promoter template comprises a 3′ terminal nucleotide extension to prevent strand displacement.

13. The method of claim 1, wherein the single stranded promoter template is composed solely of RNA.

14. The method of claim 1, wherein the single stranded promoter template is composed solely of DNA.

15. The method of claim 1, wherein the single stranded promoter template is composed of both RNA and DNA.

16. The method of claim 15, wherein the DNA portion of the single stranded promoter template hybridizes to the exposed 3′ single stranded DNA tail on the cDNA molecule or molecules and the RNA portion of said promoter template remains unhybridized.

17. The method of claim 1, wherein the exposed 3′ single stranded DNA tail is extending using a reverse transcriptase and a DNA polymerase.

18. The method of claim 1, wherein steps c) through g) are performed substantially at the same time.

19. The method of claim 1, further comprising reverse transcribing the resulting sRNA molecule or molecules, thereby producing a single stranded cDNA molecule or molecules.

20. The method of claim 1, further comprising adding a polyA tail to the resulting sRNA molecule or molecules.

21. A method for probing a nucleic acid microarray, comprising: contacting a nucleic acid microarray with the cDNA molecule or molecules of claim 16.

22. A method for synthesizing at least one sRNA molecule, comprising:
(a) providing at least one single stranded cDNA molecule having a 5′ end and a 3′ end;
(b) annealing to the 3′ end of said cDNA molecule a single stranded RNA/DNA composite bridge oligonucleotide comprising a 5′ RNA portion and a 3′ DNA sequence portion, such that the RNA portion remains single stranded;
(d) extending the 3′ end of said cDNA molecule, such that said single stranded RNA portion becomes a double stranded RNA/DNA duplex;
(e) degrading the RNA portion of said RNA/DNA duplex, thereby exposing a 3′ single stranded DNA tail;
(f) annealing to said 3′ single stranded DNA tail a single stranded promoter template comprising at least one RNA polymerase recognition sequence;
(g) extending said 3′ single stranded DNA tail such that said at least one single stranded RNA polymerase promoter template is converted into at least one RNA polymerase promoter;
(h) and initiating RNA transcription using an RNA polymerase which recognizes said at least one RNA polymerase promoter,
thereby synthesizing at least one sRNA molecule.

23. A method for performing multiple rounds of synthesis of at least one sRNA molecule, comprising:
(a) providing at least one first round single stranded cDNA molecule having a 5′ end and a 3′ end;
(b) attaching an oligodeoxynucleotide tail onto the 3′ end of said first round cDNA molecule;
(c) annealing to said oligodeoxynucleotide tail a single stranded RNA/DNA composite bridge oligonucleotide comprising a 5′ RNA portion and a 3′ DNA portion, such that the RNA portion remains single stranded;
(d) extending the 3′ end of said oligodeoxynucleotide tail, such that said single stranded RNA portion becomes a double stranded RNA/DNA duplex;
(e) degrading the RNA portion of said RNA/DNA duplex, thereby exposing a 3′ single stranded DNA tail;
(f) annealing to said 3′ single stranded DNA tail a single stranded promoter template comprising a first RNA polymerase recognition sequence and at least a second different RNA polymerase recognition sequence 3′ to said first recognition sequence;
(g) extending said 3′ single stranded DNA tail such that said single stranded promoter template is converted into a first RNA polymerase promoter and at least a second RNA polymerase promoter 3′ to said first promoter;
(h) initiating a first round of RNA transcription using an RNA polymerase which recognizes said first RNA polymerase promoter to produce at least one first round sRNA molecule;
(i) synthesizing at least one second round single stranded cDNA molecule having 5′ and 3′ ends from said first round sRNA molecule, thereby forming a double stranded sRNA/cDNA duplex;
(j) degrading the sRNA portion of said sRNA/cDNA duplex leaving said second round single stranded cDNA molecule;
(k) annealing a single stranded promoter oligonucleotide complementary to said second different RNA polymerase recognition sequence of said second round single stranded cDNA molecule such that a second RNA polymerase promoter is formed;
(l) and initiating a second round of RNA transcription using an RNA polymerase which recognizes said second RNA polymerase promoter to produce at least one second round sRNA molecule,
thereby performing multiple rounds of synthesis of at least one sRNA molecule.

24. A kit for synthesizing at least one sRNA molecule, comprising: a single stranded promoter template comprising at least one RNA polymerase recognition sequence; a single stranded RNA/DNA composite bridge oligonucleotide comprising a 5′ RNA portion and a 3′ DNA portion; and instructional materials for synthesizing sRNA molecules using said promoter template and said RNA/DNA composite bridge oligonucleotide.

25. A kit for performing multiple rounds of synthesis of at least one sRNA molecule, comprising: a single stranded promoter template comprising a first RNA polymerase recognition sequence and at least a second different RNA polymerase recognition sequence 3′ to said first recognition sequence; a single stranded RNA/DNA composite bridge oligonucleotide comprising a 5′ RNA portion and a 3′ DNA portion; a single stranded promoter oligonucleotide complementary to said second different RNA polymerase recognition sequence; and instructional materials for performing multiple rounds of synthesis of at least one sRNA molecule using said promoter template, said RNA/DNA composite bridge oligonucleotide and promoter oligonucleotide.
